SEQUENCE LISTING (3 sequences)

SEQ ID NO: 1 - protein, 659 aa, synthetic construct
  REGION 1..659: note = GLB1S protein
MPGFLVRILLLLLVLLLLGPTRGLRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEK

SEQ ID NO: 2 - protein, 677 aa, synthetic construct
  REGION 1..677: note = GLB1 protein
MPGFLVRILLLLLVLLLLGPTRGLRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 3 - protein, 232 aa, synthetic construct
  REGION 1..232: note = hinge, CH2, and CH3 of heavy chain of immunoglobulin gamma-1
EPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0132865 A1
(43) Pub. Date: Apr. 25, 2024

(54) PHARMACEUTICAL COMPOSITION FOR TREATING, PREVENTING OR AMELIORATING GM1 GANGLIOSIDOSIS OR MORQUIO SYNDROME B AND ADMINISTRATION METHOD THEREOF

(71) Applicant: NOVEL PHARMA INC., Seoul (KR)

(72) Inventor: Dong Kyu JIN, Seoul (KR)

(73) Assignee: NOVEL PHARMA INC., Seoul (KR)

(21) Appl. No.: 18/463,642

(22) Filed: Sep. 8, 2023

Related U.S. Application Data
(60) Provisional application No. 63/379,216, filed on Oct. 12, 2022.

(51) Int. Cl. C12N 9/38; A61P 25/00; C12N 5/071; C12N 15/85

(52) U.S. Cl. CPC C12N 9/2471; A61P 25/00; C12N 5/0682; C12N 15/85

(57) ABSTRACT

The present disclosure relates to a recombinant protein of beta-galactosidase-1 (GLB1) with a truncated C-terminus; a pharmaceutical composition for the treatment, prevention or amelioration of GM1 gangliosidosis or Morquio syndrome B, the pharmaceutical composition comprising the above protein; a method for treating, preventing or ameliorating GM1 gangliosidosis or Morquio syndrome B, the method comprising administering to a subject the above pharmaceutical composition; and a method of producing the above protein.

In one embodiment of the present disclosure, the recombinant protein of GLB1 with a truncated C-terminus does not pose an issue of heterogeneous protein truncation, shows an equivalent enzymatic activity compared to a full-length GLB1 protein and increases GLB1 enzyme activity in a patient's blood. The present disclosure shows that the above protein may be used to treat, prevent or ameliorate GM1 gangliosidosis or Morphio syndrome B.

### CROSS REFERENCE TO RELATED APPLICATIONS

This application claims priority to U.S. Provisional Application No. 63/379,216 filed Oct. 12, 2022, the entire disclosures of which are incorporated herein by reference.

### INCORPORATION BY REFERENCE OF SEQUENCE LISTING

The content of the electronically submitted sequence listing, file name: Q291641_sequence listing as filed; size: 6,288 bytes; and date of creation: Sep. 8, 2023, filed herewith, is incorporated herein by reference in its entirety.

### FIELD OF THE APPLICATION

The present disclosure relates to a recombinant protein of beta-galactosidase-1 (GLB1) with a truncated C-terminus; a pharmaceutical composition for the treatment, prevention or amelioration of GM1 gangliosidosis or Morquio syndrome B, the pharmaceutical composition comprising the above protein; a method for treating, preventing or ameliorating GM1 gangliosidosis or Morquio syndrome B, the method comprising administering to a subject the above pharmaceutical composition.

### BACKGROUND

GM1 gangliosidosis is a hereditary, central nervous system (CNS) disease that destroys nerve cells in the central and peripheral nerves and is caused by impaired GLB1 enzyme activity in lysosomes due to GLB1 gene mutation. The genetic cause of GM1 gangliosidosis is an autosomal, recessive mutation in the GLB1 gene, which produces beta-galactosidase-1. Due to deficient beta-galactosidase-1 enzyme activity in lysosomes, GM1 gangliosidosis prevents degradation of oligosaccharides, and eventually results in an accumulation of galactose-rich oligosaccharides and keratan sulfate in the brain and internal organs, causing the disease.

Depending on the age of onset, GM1 gangliosidosis is divided into three phenotypes: infantile type, juvenile type, and adult type. Infantile-type GM1 gangliosidosis (Type I) shows symptoms such as hepatosplenomegaly, swollen face, wide upper lips, maxillary hyperplasia, gingival hyperplasia and macroglossia. At the age of about six months, changes in the skeletal system (e.g., spine) are observed, similar to the Hurler syndrome. Juvenile-type GM1 gangliosidosis (Type II) has no externally discernible features. Patients are usually normal by the age of one, but after then, the patients lose their control function, show autistic behavior and/or become insensitive. These symptoms may develop into ataxia, epilepsy and/or spastic paralysis. The patients if untreated typically die before the age of 10, and symptoms such as vertebral breaking may occur. Adult-type GM1 gangliosidosis (Type III) does not have any externally discernible characteristics. It develops in childhood and usually shows extrapyramidal symptoms similar to symptoms of dystonia, Parkinson's disease, and non-typical cerebellar atrophy-related ataxia and serious dementia.

GM1 gangliosidosis is estimated to occur in one out of 100,000 to 200,000 newborns, and the infantile type (Type I) is reported more frequently than other phenotypes.

Morquio syndrome is an autosomal recessive, hereditary disease. Morquio syndrome type B is caused by defective degradation of keratan sulfate due to deficiency in beta-galactosidase enzyme. Symptoms of Morquio syndrome become evident around 18-24 months of age, and several complications cause patients to die primarily before the age of 20, if untreated.

Physical features of patients with Morquio syndrome include, e.g., low height, coarse face, low nasal bridge, wide nose, thick lips, macrocephaly, corneal turbidity, short neck, glaucoma, retinal degeneration and hearing loss. In addition, patients with Morquio syndrome may have skeletal dysplasia, resulting in protruded sternum, abnormal shape of the thorax and/or severe kyphosis due to spinal abnormalities, which may cause respiratory failure or spinal cord compression. Other symptoms of Morquio syndrome include inguinal hernia, hepatomegaly and dental caries due to odontoid hypoplasia may be accompanied.

Currently, there are no effective treatment methods for GM1 gangliosidosis and Morquio syndrome, and only symptomatic therapies are available. There is a need for treatment for GM1 gangliosidosis and Morquio syndrome accordingly.

Enzyme replacement therapy (ERT) is a treatment method of systemically administering a natural or recombinant protein and/or enzyme to a subject, and is considered as one of the treatments for a disease caused by enzyme defects or deficiencies.

In this context, the present disclosure relates to a recombinant protein of GLB1 with a truncated C-terminus (hereinafter referred to as “GLB1S”), and the recombinant protein shows no heterogeneous protein truncation issue and has high enzymatic activity. The present disclosure shows a surprising effect of treating, preventing or ameliorating GM1 gangliosidosis or Morquio syndrome B when the recombinant protein GLB1S is administered to a patient.

### SUMMARY

The present disclosure is related to a recombinant protein of GLB1 that has a truncated C-terminus compared to GLB1 (GLB1S). In one embodiment, the present disclosure is related to a polynucleotide encoding the recombinant protein, an expression vector comprising the polynucleotide and/or a host cell comprising the polynucleotide or the expression vector.

The present disclosure is directed to a pharmaceutical composition for treating, preventing and/or ameliorating GM1 gangliosidosis or Morquio syndrome B or at least one of their symptoms, the pharmaceutical composition comprising the above recombinant protein (GLB1S).

The present disclosure is related to a method of treatment, prevention or amelioration of GM1 gangliosidosis or Morquio syndrome B or at least one of their symptoms, the method comprising administering a pharmaceutical composition comprising said recombinant protein (GLB1S).

In one embodiment of the present disclosure, a recombinant protein of GLB1 (GLB1S) that has a truncated C-terminus does not have a protein truncation issue during the protein manufacturing process. In one embodiment, the recombinant, truncated GLB1 has an equivalent enzymatic activity compared to a full-length GLB1 protein. In one embodiment, the recombinant, truncated GLB1 has an enzymatic activity that is at least equivalent to that of a full-length GLB1 protein. In one embodiment, the short form of the recombinant GLB1 protein (GLB1S) may increase an enzymatic activity of GLB1 in patients' blood. In one embodiment of the present disclosure, the short form of the recombinant GLB1 protein (GLB1S) or a pharmaceutical composition comprising the same may be used as a drug with better efficacy, compared to the full-length GLB1 protein, for treating, preventing or ameliorating GM1 gangliosidosis or Morquio syndrome B.

The present disclosure is related to a method of producing a protein comprising an amino acid sequence of SEQ ID NO: 1, the method comprising a) introducing a target gene encoding a protein comprising an amino acid sequence of SEQ ID NO: 1 into a vector; b) transfecting a host cell with the vector; c) culturing the transfected cell and harvesting cell culture supernatants; and d) purifying the supernatant to obtain the protein.

In one embodiment of the present disclosure, the host cell is a Chinese hamster ovary (CHO) cell.

The present disclosure is related to a protein produced by said method of producing a protein comprising an amino acid sequence of SEQ ID NO: 1.

### BRIEF DESCRIPTION OF THE DRAWINGS

FIG. 1 is a detailed work flow for purification of GLB1S.

FIG. 2 shows a sodium dodecyl sulfate-polyacrylamide gel electrophoresis (SDS-PAGE) analysis result of purified recombinant GLB1S, obtained by culturing CHO-K1 cells on a 50 L scale

FIG. 3A and 3B are a size exclusion-high performance liquid chromatography (SEC-HPLC) analysis result of purified recombinant GLB1S, obtained by culturing CHO-K1 cells on a 50 L scale.

FIG. 4A and 4B are a reverse phase-high performance liquid chromatography (RP-HPLC) analysis result of purified recombinant GLB1S, obtained by culturing CHO-K1 cells on a 50 L scale.

FIG. 5 shows genotyping results of GLB1 KO mice obtained by PCR screening.

FIG. 6A to 6F show keratan sulfate content analyzed from mouse liver tissue for each group (G2 to G7) on Day 7, 14 and 28.

FIG. 7 shows keratan sulfate content analyzed from mouse liver tissue for groups G2 to G7 on Day 7.

FIG. 8 shows keratan sulfate content analyzed from mouse liver tissue for groups G2 to G7 on Day 14.

### DETAILED DESCRIPTION

Various embodiments, aspects or examples of the present disclosure are for the purpose of illustrating and/or explaining the present application and are not intended to be limiting. The present disclosure include various modifications, equivalents and alternatives of each embodiment, aspect or example described in this application and any possible combinations of all or part of each embodiment, aspect or example described in this application. One of ordinary skill in the art to which this disclosure pertains would understand and recognize said modifications, equivalents and alternatives or said combinations described above. The scope of rights of the present disclosure is not limited to various embodiments, aspects or examples set forth below or the specific descriptions of the embodiments, aspects or examples.

All technical and scientific terms used in this application, unless otherwise defined, generally have the ordinary meanings understood by a person of ordinary skill in the art to which this disclosure pertains. All terms used in this application are chosen for the purpose of describing and/or explaining the present disclosure and are not intended to limit the scope of rights under this disclosure. Certain terms are discussed in this application to provide additional guidance in describing and/or explaining the compositions and method of the present disclosure.

### Definitions

Whenever used in the present disclosure, the singular forms “a,” “an” and “the” and singular words include plural reference unless the context clearly dictates otherwise, and the same shall apply to the singular forms and words set forth in the claims. For example, “a compound” includes a plurality of such compounds, and “a compound A” includes a plurality of compounds A.

The term “and/or” means any one or more of the items, any combination of the items, or all of the items with which this term is associated. The terms “containing,” “contain,” “contains,” “including,” “include,” “includes,” “having,” “have,” “has,” “with” or variants thereof are interpreted as inclusive in a manner similar to the term “comprising.” The terms “comprising,” “including,” “having,” “containing” and variants thereof, as used in the present disclosure, are understood as open-ended terms that imply the possibility of including other embodiments, unless clearly stated otherwise. The term “consisting of” and variants thereof, as used in the present disclosure, are understood as close-ended terms that exclude any component not listed in the description of embodiments (i.e., “including and limited to”).

All ranges recited in this application may include any and all possible subranges and combinations of subranges thereof. A recited range includes each specific value, integer or decimal within the ranges. One of ordinary skill in the art may readily understand that any recited range sufficiently describes and/or enables its subranges, including and not limited to equal halves, thirds, quarters, fifths or tenths of the recited range.

The terms “greater than,” “more than,” “at least,” “or more,” “less than,” “up to” and the like, include the numbers recited, and the terms refer to ranges that may be broken down into subranges as discussed above. Specific values recited for ranges are for illustration purposes only and are not intended to be limiting, and they do not exclude other values within the ranges.

Unless indicated otherwise, the term “about” and “approximately” generally include values proximate to the recited range or value within an acceptable degree of error, as well known to those skilled in the art. The acceptable degree of error may be determined in view of the nature or precision of the measurements or the manufacturing processes. In one embodiment, the acceptable degree of error may be determined based on equivalence in terms of the functionality of the composition or method or the embodiment. In one embodiment, in the context of numerical values or ranges set forth in this disclosure, the term “about” or “approximately” can refer to a variation of ±25%, ±20%, ±15%, ±10%, ±9%, ±8%, ±7%, ±6%, ±5%, ±4%, ±3%, ±2% or ±1% of the value provided. In another embodiment, particularly in biological systems, the terms “about” and “approximately” may mean values that are within an order of magnitude, within 9-fold, 8-fold, 7-fold, 6-fold, 5-fold, 4-fold, 3-fold or 2-fold of a given value. All numerical quantities provided in this application are approximations unless stated otherwise. In addition, these quantities inherently contain variability necessarily resulting from their measurements.

In the present disclosure, the term “pharmaceutical composition” may refer to a form of a pharmaceutical preparation that allows the biological activity of an active ingredient contained in the composition to be effective. In one embodiment, the pharmaceutical composition is a composition comprising one or more active ingredients that can be administered to a patient and does not further comprise an unacceptable toxic component.

In the present disclosure, the term “pharmaceutically acceptable” may refer to a property of substance that is useful for the preparation of generally safe, non-toxic and biologically or otherwise desirable pharmaceutical compositions and is acceptable for human pharmaceutical applications and/or veterinary applications. In one embodiment, the term “pharmaceutically acceptable” means approved or approvable by a government regulatory agency or listed in generally recognized pharmacopoeia for use in humans and/or animals.

In the present disclosure, the term “pharmaceutically acceptable excipient” refers to any component that is not therapeutically active (inert) and non-toxic. In one embodiment, the pharmaceutically acceptable excipient may include, but is not limited to, for example, a binder, filler, solvent, buffer, tonicity agent, stabilizer, antioxidant, surfactant or lubricant configured to be used in formulating a pharmaceutical product.

As used herein, the term “pharmaceutically acceptable carrier” refers to a component of a pharmaceutical composition other than an active ingredient, the component being non-toxic to a subject. In one embodiment, the pharmacologically acceptable carrier may include, but is not limited to, for example, a buffer, excipient, stabilizer or preservative.

In the present disclosure, the terms “pharmaceutically effective dose,” “pharmaceutically effective amount,” “administration dose,” “administration amount,” “therapeutically effective dose,” “therapeutically effective amount,” “effective dosage” or “effective amount” of a pharmaceutical composition may refer to an amount of a pharmaceutical composition (to be administered for the required duration) that is sufficient to achieve a therapeutic response or effect, a desired local or systemic therapeutic result or a desired preventive result. In one embodiment, the above terms refer to an amount of a pharmaceutical composition that, when administered to a subject, (i) treats or prevents, (ii) mitigates, reduces, ceases, attenuates, ameliorates or eliminates one or more symptoms of, or (iii) prevents or delays the onset of one or more symptoms of the disease, condition or disorder described herein.

In the present disclosure, the term “subject” refers to a mammal. Mammals may include, but are not limited to, domesticated animals (e.g., cows, sheep, cats, dogs, and horses), primates (e.g., human and non-human primates, such as monkeys), rabbits and rodents (e.g., mice and rats). In one embodiment, the subject may be a human.

One aspect of the present disclosure is directed to a protein comprising the amino acid sequence of SEQ ID NO:1. The amino acid sequence of SEQ ID NO:1 in the present disclosure is a recombinant protein (GLB1S) that is truncated at the C-terminus of a beta-galactosidase-1 (GLB1) protein.

One aspect of the present disclosure provides a protein comprising either the amino acid sequence of SEQ ID NO:1 or an amino acid sequence having homology of at least about 80%, at least about 85%, at least about 90%, at least about 91%, at least about 92%, at least about 93%, at least about 94%, at least about 95%, at least about 96%, at least about 97%, at least about 98% or at least about 99% to the amino acid sequence of SEQ ID NO:1. In one embodiment, the protein may comprise, without limitation, may comprise any amino acid sequence having homology to the amino acid sequence of SEQ ID NO:1 and having an activity that is the same or substantially the same as, or corresponds to, that of the protein comprising the amino acid sequence of SEQ ID NO:1. In one embodiment, the protein may comprise any amino acid sequence having a deletion, modification, substitution or addition of one or more amino acids, as long as the amino acid sequence has homology to the amino acid sequence of SEQ ID NO:1; the amino acid sequence has one or more activities of the protein comprising the amino acid sequence of SEQ ID NO:1; or such deletion, modification, substitution or addition does not substantially affect the function of said protein. For example, one or more amino acids may be deleted, modified, substituted, or added for optimization of and/or convenience in the process of expression and extraction of the protein.

In the present disclosure, “homology” may refer to, in an amino acid sequence or nucleic acid sequence of a gene encoding a protein, the degree of identity of nucleic acids or amino acid residues between two sequences after aligning the two sequences to match as much as possible in a specific comparative region. If the homology between two genes is sufficiently high, the expression products of the two genes may have the same or similar activity. The percentage (%) of said sequence identity may be determined using a publicly known sequence comparison program (e.g., Blast (NCBI)).

In one embodiment of the present application, proteins can be expressed and produced by methods known in the art to which this disclosure pertains. Host cells suitable for expressing and producing proteins described in this disclosure include and are not limited to bacterial cells such as E. coli, Streptomyces and Salmonella typhimurium, yeast cells such as Saccharomyces cerevisiae, Schizosaccharomyces pombe, Pichia pastoris; insect cells such as Drosophila, Spodoptera Sf9 cells; animal cells such as CHO, COS, NSO, 293, Bowes melanoma cells; or plant cells.

In one embodiment, host cells suitable for expressing and producing proteins described in this disclosure include vertebrate host cells and eukaryotic cells as described herein. Examples of mammal host cell lines include, but are not limited to, monkey kidney cells (CV1), monkey kidney CV1 cells transformed by SV40 (COS-7), human embryonic kidney cells (293 cells), baby hamster kidney cells (BHK), Chinese Hamster ovarian cells (CHO), mouse Sertoli cells (TM4), African green monkey kidney cells (VERO-76), human cervical carcinoma cells (HELA), dog kidney cells (MDCK), buffalo rat liver cells (BRL 3A), human lung cells (W138), human hepatocytes (Hep G2), mouse breast tumors (MMT 060562), TRI cells, MRC 5 cell, and FS4 cells.

In one embodiment, host cells used to produce the proteins described in the present disclosure can be cultured in various media. Commercially available media, including and not limited to, e.g., Ham F10 medium, minimum essential medium (MEM), RPMI-1640, Dulbecco's Modified Eagle Medium (DMEM) and any other medium used in the art to which this disclosure pertains, may be used for host cell culture. Any of these media may be supplemented with one or more hormones and/or growth factors (e.g., insulin, transferrin or epidermal growth factors), one or more salts (e.g., sodium chloride, calcium, magnesium or phosphate), one or more buffers (e.g., HEPES), one or more nucleotides (e.g., adenosine or thymidine), one or more antibiotics (e.g., Gentamycin™), one or more trace elements, glucose and/or one or more equivalent energy sources, if necessary. Any other essential/auxiliary supplements may also be included in appropriate concentrations known to a person skilled in the art. Other culture conditions, including and not limited to, e.g., temperature, pH and CO₂ concentration, can be set to suitable conditions known to a person skilled in the art in relation to host cells selected for protein expression.

In one embodiment, the proteins described in the present disclosure can be purified in any suitable protein purification method known in the art to which this disclosure pertains. Examples of known protein purification methods include, and are not limited to, hydrophobic interaction chromatography (HIC), fractionation on an immunoaffinity or ion-exchange column, ethanol precipitation, reverse phase HPLC, chromatography on silica, chromatography on an anion-exchange resin or a cation-exchange resin, chromatofocusing, SDS-PAGE, ammonium sulfate precipitation, gel filtration, genetic engineering methods (e.g., tag) and/or other similar purification methods, and any combinations thereof (e.g., to increase the purity of the proteins). Proteins purified via the above methods in the present disclosure may have a high degree of purity that can be administered to animals, particularly humans, for experimental, clinical and therapeutic purposes.

According to one embodiment of the present disclosure, a protein comprising the amino acid sequence of SEQ ID NO:1 (GLB1S protein) shows GLB1 activity without protein truncation issues in high purity. In addition, the enzyme activity of the GLB1S protein is at least equivalent to that of the full-length GLB1 protein, as the enzyme activity of the GLB1S protein is higher by about 10% to about 20% than that of the full-length GLB1 protein. The enzyme activity was measured by a beta-galactosidase activity assay, as known in the art. Specifically, the enzyme activity was measured by detecting the fluorescence of 4-methylumbelliferone (4-MU), generated when the substrate 4-methylumbelliferyl-β-galactopyranoside (MUG) is cleaved by the GLB1 or GLB1S enzyme.

The above protein truncation issue refers to elimination of the N- or C-terminal portion of a protein. In case of GLB1, random cleavage is observed at the C-terminal (all or a portion of R637 to V654 in the amino acid sequence of the full-length GLB1 protein (comprising the amino acid sequence of SEQ ID NO:2) upon protein expression. If variants with different C-terminal sequences are produced, problems may arise in terms of quality control and, potentially, the drug approval process. For example, during the approval process, questions may arise including, e.g., (i) whether there is a difference in efficacy between the variants, (ii) whether there is a difference in stability and/or effect if the ratio of the variants in the drug product varies and (iii) how the ratio of the variants in the drug product can be uniformly managed. The GLB1S protein comprising the amino acid sequence of SEQ ID NO:1 of the present disclosure can resolve these issues.

| SEQ ID NO: 1 | |
|---|---|
| 1 | MPGFLVRILL LLLVLLLLGP TRGLRNATQR MFEIDYSRDS FLKDGQPFRY ISGSIHYSRV |
| 61 | PRFYWKDRLL KMKMAGLNAI QTYVPWNFHE PWPGQYQFSE DHDVEYFLRL AHELGLLVIL |
| 121 | RPGPYICAEW EMGGLPAWLL EKESILLRSS DPDYLAAVDK WLGVLLPKMK PLLYQNGGPV |
| 181 | ITVQVENEYG SYFACDFDYL RFLQKRFRHH LGDDVVLFTT DGAHKTFLKC GALQGLYTTV |
| 241 | DFGTGSNITD AFLSQRKCEP KGPLINSEFY TGWLDHWGQP HSTIKTEAVA SSLYDILARG |
| 301 | ASVNLYMFIG GTNFAYWNGA NSPYAAQPTS YDYDAPLSEA GDLTEKYFAL RNIIQKFEKV |
| 361 | PEGPIPPSTP KFAYGKVTLE KLKTVGAALD ILCPSGPIKS LYPLTFIQVK QHYGFVLYRT |
| 421 | TLPQDCSNPA PLSSPLNGVH DRAYVAVDGI PQGVLERNNV ITLNITGKAG ATLDLLVENM |
| 481 | GRVNYGAYIN DFKGLVSNLT LSSNILTDWT IFPLDTEDAV RSHLGGWGHR DSGHHDEAWA |
| 541 | HNSSNYTLPA FYMGNFSIPS GIPDLPQDTF IQFPGWTKGQ VWINGENLGR YWPARGPQLT |
| 601 | LFVPQHILMT SAPNTITVLE LEWAPCSSDD PELCAVTFVD RPVIGSSVTY DHPSKPVEK |
| SEQ ID NO: 2 | |
| 1 | MPGFLVRILL LLLVLLLLGP TRGLRNATQR MFEIDYSRDS FLKDGQPFRY ISGSIHYSRV |
| 61 | PRFYWKDRLL KMKMAGLNAI QTYVPWNFHE PWPGQYQFSE DHDVEYFLRL AHELGLLVIL |
| 121 | RPGPYICAEW EMGGLPAWLL EKESILLRSS DPDYLAAVDK WLGVLLPKMK PLLYQNGGPV |
| 181 | ITVQVENEYG SYFACDFDYL RFLQKRFRHH LGDDVVLFTT DGAHKTFLKC GALQGLYTTV |
| 241 | DFGTGSNITD AFLSQRKCEP KGPLINSEFY TGWLDHWGQP HSTIKTEAVA SSLYDILARG |
| 301 | ASVNLYMFIG GTNFAYWNGA NSPYAAQPTS YDYDAPLSEA GDLTEKYFAL RNIIQKFEKV |
| 361 | PEGPIPPSTP KFAYGKVTLE KLKTVGAALD ILCPSGPIKS LYPLTFIQVK QHYGFVLYRT |
| 421 | TLPQDCSNPA PLSSPLNGVH DRAYVAVDGI PQGVLERNNV ITLNITGKAG ATLDLLVENM |
| 481 | GRVNYGAYIN DFKGLVSNLT LSSNILTDWT IFPLDTEDAV RSHLGGWGHR DSGHHDEAWA |
| 541 | HNSSNYTLPA FYMGNFSIPS GIPDLPQDTF IQFPGWTKGQ VWINGENLGR YWPARGPQLT |
| 601 | LFVPQHILMT SAPNTITVLE LEWAPCSSDD PELCAVTFVD RPVIGSSVTY DHPSKPVEKR LMPPPPQKNK DSWLDHV |

In order to solve the above issues, a method such as treating the full-length GLB1 protein with trypsin to remove the irregular C-terminus may be considered, but the method has problems such as significantly low yield of protein production, excessive cost and incomplete purification. According to one embodiment of the present invention, GLB1S protein comprising the amino acid sequence of SEQ ID NO:1 does not require the use of proteolytic enzyme (e.g., trypsin), so the protein purification of GLB1S is easier, and the protein with high purity may be obtained.

Another aspect of the present disclosure provides a polynucleotide encoding a protein comprising the amino acid sequence of SEQ ID NO:1. According to one embodiment of the present disclosure, the nucleic acid sequence comprising the above polynucleotide may be in the form of a nucleic acid sequence encoding the amino acid sequence of SEQ ID NO:1. In another embodiment of the present disclosure, one or more nucleic acid sequences encoding various amino acid sequences that could be added to the N-terminus or C-terminus of the amino acid sequence of SEQ ID NO:1 may be added to the 5′-terminus or 3′-terminus of the nucleic acid sequence encoding the amino acid sequence of SEQ ID NO:1.

In one embodiment of the present disclosure, the above polynucleotide may be modified by substitution, deletion or insertion of one or more nucleic acids or any combination thereof. If nucleotide sequences are produced by chemical synthesis, one or more synthesis methods well known in the may be used, including and not limited to, e.g., methods described in Engels and Uhlmann, Angew Chem IntEd Engl., 37:73-127, 1988; triester, phosphite, phosphoramidite and H-phosphate methods; PCR and other autoprimer methods; and oligonucleotide synthesis on solid supports.

One aspect of the present disclosure provides an expression vector comprising the above polynucleotide encoding a protein comprising the amino acid sequence of SEQ ID NO:1. The term “expression vector” in the present disclosure refers to a gene construct comprising a gene insert to express a protein of interest in appropriate host cells, and comprising essential regulatory elements operably linked such that the gene insert is expressed in the host cells.

In the present disclosure, a “vector” refers to any vehicle for the cloning of and/or transfer of a nucleic acid into a host cell. A vector may be a replicon to which another DNA segment may be attached to bring about the replication of the attached segment. A “replicon” refers to any genetic element (e.g., plasmid, phage, cosmid, chromosome, virus) that functions as an autonomous unit of DNA replication in vivo (i.e., capable of replication under its own control). The term “vector” may include both viral and non-viral vehicles for introducing the nucleic acid into an organism (e.g., a host cell) in vitro, ex vivo or in vivo. The term “vector” may also include minicircle DNAs, transposons such as Sleeping Beauty (Izsvak et al. J. Mol. Biol. 302:93-102 (2000)) or artificial chromosomes.

Examples of conventional vectors may include a natural or recombinant plasmid, cosmid, virus and bacteriophage. For example, pWE15, M13, MBL3, MBL4, IXII, ASHII, APII, t10, t11, Charon4A, and Charon21A may be used as a phage vector or cosmid vector without limitation. As a plasmid vector, pBR type, pUC type, pBluescriptII type, pGEM type, pTZ type, pCL type and pET type may be used without limitation.

The above expression vector includes regulatory elements such as a start codon, a stop codon, a promoter and an operator. The start and stop codons are generally considered part of a nucleotide sequence encoding a polypeptide and must act in an individual to whom a gene construct is administered and be in frame with the coding sequence. The promoter of the vector may be constitutive or inducible.

As used throughout the present disclosure, the term “operably linked” refers to a functional linkage between a nucleic acid sequence encoding a protein or RNA of interest and an expression regulatory sequence such that the sequences encode a desired function. For example, a nucleic acid sequence for a protein or RNA of interest is operably linked with its promoter and/or regulatory sequences such that the linked promoter and/or regulatory sequences functionally controls expression of the coding sequence. Operably linked elements may be contiguous or non-contiguous. The operable linkage with an expression vector may be using genetic recombination techniques well known in the art, and enzymes generally known in the art may be used for site-specific DNA cleavage and ligation.

In one embodiment of the present disclosure, the expression vector may introduce a polynucleotide encoding a protein comprising an amino acid sequence of SEQ ID NO:1 into CHO-K1 (CHO cell line) to transform CHO-K1 into a cell line capable of producing the above protein. Another aspect of the present disclosure provides a host cell comprising the above polynucleotide or the above expression vector.

The expression vector can be introduced into a host cell, and then the host cell can be transformed to express one or more polynucleotides included in the expression vector and produce proteins of the present disclosure. Host cells into which the above expression vector may be introduced include and are not limited to those mentioned above, as long as they can express the above polynucleotides to produce proteins of the present disclosure.

Cell transformation may be performed through a variety of methods. In one embodiment of the present disclosure, the transformation methods include, and are not limited to, the CaCl₂ precipitation method; the Hanahan's method, which increases efficiency by using a reducing agent DMSO to the CaCl₂ precipitation method; the heat shock method; the electroporation method; the calcium phosphate precipitation method; the protoplast fusion method; the agitation method using silicon carbide fibers/whiskers; the Agrobacteria-mediated transformation (AMT) method; the polyethylene glycol (PEG)-mediated transformation method; the dextran sulfate-mediated transformation method; the lipofectamine-mediated transformation method; the drying/suppression mediated transformation method and other transformation methods that enable host cells to produce the proteins of the present disclosure.

One aspect of the present disclosure provides a pharmaceutical composition for the treatment, prevention or amelioration of GM1 gangliosidosis or Morquio Syndrome B comprising a protein comprising the amino acid sequence of SEQ ID NO:1. One aspect of the present disclosure provides a method for the treatment, prevention or amelioration of GM1 gangliosidosis or Morquio Syndrome B.

As used in the present disclosure, the terms “ameliorating” or “amelioration” of a disease state may refer to any act of reducing the degree of at least one parameter (e.g., a symptom) associated with the disease state. In the context of GM1 gangliosidosis or Morquio syndrome B, the terms “ameliorating” or “amelioration” of the diseases may include any act of reducing the degree of at least one parameter associated with GM1 gangliosidosis or Morquio syndrome B by administering the above pharmaceutical composition. The terms “prevention” or “preventing” of a disease state may refer to a reduction in risk of acquiring a disease, condition or disorder or causing at least one of the clinical symptoms of the disease not to develop in a subject that may be exposed to or predisposed to the disease but does not yet experience or display symptoms of the disease. The terms “prevention” or “preventing” of GM1 gangliosidosis or Morquio syndrome B in the present disclosure may include any act of inhibiting or delaying the onset of GM1 gangliosidosis or Morquio syndrome B or causing at least one of the clinical symptoms of the disease not to develop in a subject by administering said pharmaceutical composition. The terms “treat,” “treating” or “treatment,” as used herein, include alleviating, abating or ameliorating a disease, condition or disorder or its symptoms, preventing additional symptoms, ameliorating or preventing the underlying causes of symptoms, inhibiting the disease, condition or disorder (e.g., arresting the development of the disease, condition or disorder), relieving the disease, condition or disorder, causing regression of the disease, condition or disorder, relieving a condition caused by the disease, condition or disorder, or stopping the symptoms of the disease, condition or disorder either prophylactically and/or therapeutically. The term “treatment” of GM1 gangliosidosis or Morquio syndrome B may include any act of alleviating, abating or ameliorating GM1 gangliosidosis or Morquio syndrome B or symptoms of the disease that developed or are suspected to have developed in a subject by administering said pharmaceutical composition.

The pharmaceutical composition of the present disclosure may comprise a pharmaceutically effective amount of the protein of the present disclosure, and may further comprise a pharmaceutically acceptable carrier.

In the present disclosure, GM1 gangliosidosis refers to a hereditary, CNS disease that destroys nerve cells in the central and peripheral nerves and is caused by a deficiency in beta-galactosidase-1 enzyme activity in lysosomes due to GLB1 gene mutation. Morquio Syndrome B in the present disclosure refers to a disease caused by an accumulation of keratan sulfate due to a deficiency in beta-galactosidase enzyme and a subsequent defective degradation of keratan sulfate.

In one embodiment, the protein comprising the amino acid sequence of SEQ ID NO:1 may be included in a pharmaceutical composition at a concentration of, without limitation, about 0.001 to about 500 mg/ml, at a concentration of about 1 to about 50 mg/ml, at a concentration of about 1 to about 10 mg/ml, at a concentration of about 3 to about 7 mg/ml, at a concentration of about 5 mg/ml, at a concentration of about 10 to about 50 mg/ml, at a concentration of about 20 to about 40 mg/ml, or at a concentration of about 30 mg/ml. In one embodiment, the protein comprising the amino acid sequence of SEQ ID NO:1 may be included in a pharmaceutical composition administered intravenously at a concentration of, without limitation, about 1 to about 10 mg/ml. In one embodiment, the protein comprising the amino acid sequence of SEQ ID NO:1 may be included in a pharmaceutical composition administered intracerebroventricularly at a concentration of, without limitation, about 10 to about 50 mg/ml.

In one embodiment of the present disclosure, a pharmaceutical composition comprising the above protein can be formulated and administered in a manner consistent with medical and clinical practice. Factors considered in this regard include, but are not limited to, the disease being treated, the specific animal being treated, the clinical condition of an individual subject, the cause of the disease, the delivery site of the substance, the method of administration, the administration plan and other factors known to clinicians. In one embodiment, formulations of the pharmaceutical composition may include, but are not limited to, a liquid formulation or a freeze dry powder formulation. In one embodiment, the pharmaceutical composition of the present disclosure can be manufactured in the form of an ampoule, a vial, a bottle, a cartridge, a reservoir, a lyo-ject or a pre-filled syringe. In one embodiment, the pharmaceutical composition may be manufactured in a single dosage form or a multiple dosage form.

The terms “pharmaceutically effective dose,” “pharmaceutically effective amount,” “administration dose,” “administration amount,” “therapeutically effective dose,” “therapeutically effective amount,” “effective dose” and/or “effective amount” of the protein of the present disclosure administered to a subject refer to the minimum amount required for the prevention, amelioration or treatment of a particular disease (e.g., GM1 gangliosidosis or Morquio syndrome B) and may be determined by the considerations described above. In one embodiment, the terms “pharmaceutically effective dose,” “pharmaceutically effective amount,” “administration dose,” “administration amount,” “therapeutically effective dose,” “therapeutically effective amount,” “effective dose” and/or “effective amount” of the above protein may be, but are not limited to, e.g., from about 0.0001 mg/kg to about 100 mg/kg per dose, from about 0.001 mg/kg to about 100 mg/kg per dose, from about 0.01 mg/kg to about 100 mg/kg per dose, from about 0.1 mg/kg to about 100 mg/kg per dose, from about 0.1 mg/kg to about 30 mg/kg per dose, from about 0.1 mg/kg to about 10 mg/kg per dose, from about 1 mg/kg to about 30 mg/kg per dose, or from about 1 mg/kg to about 5 mg/kg. Accordingly, the pharmaceutical composition of the present disclosure may include from about 0.003 to about 3000 mg, from about 30 mg to about 900 mg, or from about 30 mg to about 150 mg of the protein of the present disclosure (e.g., GLB1S protein) in a single dose. In one embodiment, the pharmaceutical composition of the present disclosure may be administered intravenously at a concentration of, without limitation, about 0.1 mg/kg/week to about 10 mg/kg/week, about 1 mg/kg/week to about 5 mg/kg/week or about 2 mg/kg/week. In one embodiment, the pharmaceutical composition of the present disclosure may be administered intracerebroventricularly at concentration of, without limitation, about 5 mg/head/biweekly to about 30 mg/head/biweekly, about 15 mg/head/biweekly to about 30 mg/head/biweekly, about 15 mg/head/biweekly, about 5 mg/head/monthly to about 30 mg/head/monthly, about 15 mg/head/monthly to about 30 mg/head/monthly, or about 15 mg/head/monthly.

The pharmaceutical composition of the present disclosure may be administered periodically (including but not limited to, e.g., once a day, three times a week, twice a week, once a week, once every two weeks, three times a month, twice a month or once a month), depending on the judgment of an experienced clinical practitioner, and may be administered non-periodically in situations including but not limited to, e.g., acute progression of the disease.

In one embodiment of the present application, the pharmaceutical composition of the present disclosure may be prepared using standard methods known to a person skilled in the art by, e.g., mixing the protein with a desired degree of purity with a pharmaceutically or physiologically acceptable carrier, excipient or stabilizer. In one embodiment, an acceptable carrier includes, but is not limited to, e.g., saline or buffer (e.g., phosphate, citrate and other organic acids); antioxidant (e.g., ascorbic acid); a low molecular weight polypeptide (including less than about 10 amino acid residues); protein (e.g., serum albumin, gelatin or immunoglobulin); hydrophilic polymer (e.g. polyvinylpyrrolidone); amino acid (e.g., glycine, glutamine, asparagine, arginine or lysine); monosaccharide, disaccharide and other carbohydrates (e.g., glucose, mannose or dextrin); chelating agent (e.g., EDTA); sugar alcohol (e.g., mannitol or sorbitol); salt-forming counter-ions (e.g., sodium); and/or non-ionic surfactants (e.g., TWEEN™, PLURONICS™ or PEG).

In one embodiment, the pharmaceutical composition of the present disclosure may contain a pharmaceutically acceptable salt in an approximately physiological concentration. Optionally, a formulation of the pharmaceutical composition according to one embodiment of the present disclosure may contain a pharmaceutically acceptable preservative. Specifically, in one embodiment, the preservative concentration may be from about 0.1% to about 2.0% (typically v/v). In one embodiment, the preservative may be a preservative generally known in the pharmaceutical industry, and the preservative may be, and is not limited to, e.g., benzyl alcohol, phenol, m-cresol, methylparaben, propylparaben or any combination thereof. In one embodiment, the pharmaceutical composition may contain a pharmaceutically acceptable surfactant. Specifically, in one embodiment, the surfactant concentration may be from about 0.005% to about 0.02%.

The pharmaceutical composition in the present disclosure may further comprise one or more active compounds necessary for the treatment, prevention or amelioration of GM1 gangliosidosis or Morquio syndrome B. Specifically, the pharmaceutical composition may further comprise one or more active compounds having complementary activities that do not adversely affect, and/or are not affected by, the protein of the present disclosure. The pharmaceutical composition may comprise an effective amount of the active compounds to achieve the intended purpose of the pharmaceutical composition.

In one embodiment of the present disclosure, the pharmaceutical composition may be administered to a human or animal subject via intracerebroventricular (ICV), intravenous (IV), intramuscular (IM), intraperitoneal (IP), intracerebrospinal, subcutaneous (SC), intra-articular, intrasynovial, intrathecal, oral, topical, or inhalation routes according to one or more methods generally known in the art. In one embodiment, the pharmaceutical composition may be administered ICV or IV. Specifically, the pharmaceutical composition may be administered ICV via an intraventricular catheter system comprising a reservoir and a catheter connected to the reservoir.

According to an example of the present disclosure, when the pharmaceutical composition described in this application is administered to a patient, the GLB1 enzyme activity in the patient's blood may increase by at least 50%.

According to an example of the present disclosure, when the pharmaceutical composition described in this application is administered to a patient, the amount of keratan sulfate in one or more internal organs such as brain, liver, etc., of the patient may decrease.

One aspect of the present disclosure provides a method of producing a protein comprising an amino acid sequence of SEQ ID NO: 1. In one embodiment the method comprises a) introducing a target gene encoding a protein comprising an amino acid sequence of SEQ ID NO: 1 into a vector; b) transfecting a host cell with the vector; c) culturing the transfected cell and harvesting cell culture supernatants; and d) purifying the supernatant to obtain the protein. In one embodiment, the host cell is a Chinese hamster ovary (CHO) cell.

One aspect of the present disclosure provides a protein produced by the method of producing a protein comprising an amino acid sequence of SEQ ID NO: 1.

Working and experimental examples described herein explain the composition, the method, and effects of the present disclosure in great details, but these examples are provided for illustrative purposes only, to help understand the present disclosure, and the categories and/or scope of this disclosure are not limited by the examples.

### Comparative Example (Control) 1. Production of Expression Vectors for Full-Length Recombinant Beta-Galactosidase (GLB1) Protein

Target gene, i.e., GLB1 (SEQ ID NO: 2), was cloned into mammalian expression vector to construct the plasmids. The target gene was synthesized and inserted into the upstream MCS region of the pGenHT1.0-DGV vector. pGenHT1.0-DGV is a mammalian expression vector for CHO GS system which was designed and synthesized by GenScript. The vector contains MCS for the expression of target gene of a protein. The vector contains a Glutamine Synthetase (GS) gene for the selection of positive clones. Finally, the plasmids were prepared at a concentration of 1 μg/μl.

### Comparative Example (Control) 2. GLB1 Protein Expressing Cell Line Construction and Expression Process Development of GLB1 Protein

CHOK1-GenS, which is licensed from ECACC (European Collection of Authenticated Cell Cultures), was used as a host cell. The plasmids as described in Comparative Example 1 above were transfected into CHOK1-GenS cells. At 48 hours post-transfection, the supernatants were harvested for Western blot test. The transfected cells were seeded into 24-well plates with the selection medium for pool screening. After screening, the supernatants of all cell pools were analyzed by dot blot. Based on the signal intensity, mix cell pool M1 and M2 were selected for single cell clone screening with the method of limiting dilution. Cell Metric™ CLD Imager was used to record and confirm the monoclonality of single cell clones. 43 single clones with higher titer and good confluency were selected. Finally, top clone showing the best productivity and growth performance was selected.

The selected cells were sub-cultured every 72 hours. The total passage number, i.e., the number of times the culture has been sub-cultured, was 20, and passages were named as P0-P20, respectively. The cells at P0, P17 and P20 were recovered for fed-batch culture. The cells at P0 and P20 were collected for target sequence detection. The cell growth viable cell density, viability, GLB1 expression titer and corresponding protein quality proved that the top clone was stable for over 60 days of passage.

Supernatants from P0 fed-batch cultures were harvested on the last day for purification and sequence analysis. Specifically, the C-terminal sequences of proteins were analyzed by mass spectrometry (MS). Tables 1-3 shows the MS analysis results of the C-terminal sequences of the GLB1 proteins.

TABLE 1
MS analysis results of the C-terminal sequences of GLB1 proteins produced from Clone
#1

| Peptide | | Delta | Confidence | ID | | | Charge | Mono Mass | Avg Mass | Mono Mass | | |
|---|---|---|---|---|---|---|---|---|---|---|---|---|
| Sequence | Modification | (ppm) | Score | Type | RT | M/Z | State | Exp. | Exp. | Theo. | Emzyme | Percentage |
| LMPPPQ | nonspecific | −2.12 | 100 | MS2 | 24.6 | 779.41 | 1 | 778.4031 | 778.71 | 778.4047 | Lys- | 41.19% |
| LMPPPQK | nonspecific | −0.81 | 100 | MS2 | 22.56 | 454.257 | 2 | 906.499 | 907.02 | 906.4997 | C + PNGase | 9.82% |
| LMPPPQKN | nonspecific | −0.84 | 100 | MS2 | 21.84 | 511.278 | 2 | 1020.5417 | 1021.11 | 1020.5426 | F | 25.33% |
| LMPPPQKNK | nonspecific | −2.13 | 100 | MS2 | 20.45 | 383.886 | 3 | 1148.6351 | 1149.26 | 1148.6376 | | 4.71% |
| LMPPPQKNK | nonspecific | −1.73 | 100 | MS2 | 20.73 | 422.228 | 3 | 1263.6624 | 1264.32 | 1263.6645 | | 14.93% |
| D | | | | | | | | | | | | |
| LMPPPQKNK | nonspecific | −0.96 | 100 | MS2 | 31.04 | 635.323 | 3 | 1901.944 | 1903.02 | 1901.9458 | | 2.93% |
| DSWLDH | | | | | | | | | | | | |
| LMPPPQKNK | None | 0.58 | 100 | MS2 | 35.94 | 501.512 | 4 | 2001.0154 | 2002.1 | 2001.0142 | | 0.12% |
| DSWLDHV | | | | | | | | | | | | |

TABLE 2
MS analysis results of the C-terminal sequences of GLB1 proteins produced from Clone
#2

| Peptide | | Delta | Confidence | | | | Charge | Mono Mass | Avg Mass | Mono Mass | | |
|---|---|---|---|---|---|---|---|---|---|---|---|---|
| Sequence | Modification | (ppm) | Score | Type | RT | M/Z | State | Exp. | Exp. | Theo. | Emzyme | Percentage |
| LMPPPQ | nonspecific | −1.02 | 100 | MS2 | 24.71 | 390.209 | 2 | 778.4039 | 778.71 | 778.4047 | Lys- | 42.54% |
| LMPPPQK | nonspecific | −0.34 | 100 | MS2 | 22.61 | 454.257 | 2 | 906.4994 | 907.02 | 906.4997 | C + PNGase | 10.98% |
| LMPPPQKN | nonspecific | 0 | 100 | MS2 | 21.93 | 511.279 | 2 | 1020.5426 | 1021.11 | 1020.5426 | F | 23.80% |
| LMPPPQKNK | nonspecific | −1.49 | 100 | MS2 | 20.51 | 383.886 | 3 | 1148.6359 | 1149.26 | 1148.6376 | | 6.97% |
| LMPPPQKNK | nonspecific | −0.96 | 100 | MS2 | 20.82 | 422.228 | 3 | 1263.6633 | 1264.32 | 1263.6645 | | 12.49% |
| D | | | | | | | | | | | | |
| LMPPPQKNK | None | 1.31 | 100 | MS2 | 36.05 | 501.512 | 4 | 2001.0168 | 2002.14 | 2001.0142 | | 0.22% |
| DSWLDHV | | | | | | | | | | | | |

TABLE 3
MS analysis results of the C-terminal sequences of GLB1 proteins produced from Clone
#3

| Peptide | | Delta | Confidence | ID | | | Charge | Mono Mass | Avg Mass | Mono Mass | | |
|---|---|---|---|---|---|---|---|---|---|---|---|---|
| Sequence | Modification | (ppm) | Score | Type | RT | M/Z | State | Exp. | Exp. | Theo. | Emzyme | Percentage |
| LMPPPQ | nonspecific | −0.47 | 100 | MS2 | 24.59 | 390.209 | 2 | 778.4044 | 778.71 | 778.4047 | Lys- | 32.40% |
| LMPPPQK | nonspecific | 0.19 | 100 | MS2 | 22.5 | 454.257 | 2 | 906.4999 | 907.02 | 906.4997 | C + PNGase | 11.07% |
| LMPPPQKN | nonspecific | 0.15 | 100 | MS2 | 21.81 | 511.279 | 2 | 1020.5428 | 1021.11 | 1020.5426 | F | 23.97% |
| LMPPPQKNK | nonspecific | −1.81 | 100 | MS2 | 20.35 | 383.886 | 3 | 1148.6355 | 1149.25 | 1148.6376 | | 7.97% |
| LMPPPQKNK | nonspecific | −1.25 | 100 | MS2 | 20.6 | 422.228 | 3 | 1263.663 | 1264.32 | 1263.6645 | | 18.89% |
| D | | | | | | | | | | | | |
| LMPPPQKNK | nonspecific | 0.64 | 100 | MS2 | 30.96 | 476.744 | 4 | 1901.947 | 1903.02 | 1901.9458 | | 4.09% |
| DSWLDH | | | | | | | | | | | | |
| LMPPPQKNK | None | 0.83 | 100 | MS2 | 35.88 | 501.512 | 4 | 2001.0159 | 2002.11 | 2001.0142 | | 0.29% |
| DSWLDHV | | | | | | | | | | | | |

As shown in Tables 1-3, serious C-terminal truncations of GLB1 protein were observed, in a pattern of gradual cleavage with different length of C-term amino acids. In all three top clones from cell line development, 3-4 variants with a percentage of greater than 10% were discovered, and there was no variant that could be considered as a main product. This observation intrigued the subsequent GLB1S project where artificial removal of C-term could possibly salvage the situation.

### Example 1. Plasmids Preparation for Recombinant Beta-Galactosidase (GLB1S)

The target gene, i.e., GLB1S (SEQ ID NO: 1), was optimized towards CHO host cells and was synthesized. The target gene was inserted into the upstream MCS region of the pGenHT1.0-DGV vector. The plasmids were prepared at a concentration of 1 μg/μl. The transfection plasmid was named as NP4K_pGenHT1.0-DGV.

### Example 2. GLB1S Protein Expressing Cell Line Construction and Expression Process Development of GLB1S Protein

The plasmid prepared as described in Example 1 above was transfected into CHOK1-GenS host cell. At 48 hours post-transfection, the supernatants were harvested for Fluorescent β-Galactosidase Assay and Western blot test. The transfected cells were seeded into 24-well plates with the selection medium for cell pool screening. After screening, the supernatants of all cell pools were analyzed by Dot-blot. Based on the signal intensities, mix cell pool M1, M2, M3, and M4 were selected for single cell clone screening with the method of limiting dilution. Cell Metric™ CLD Imager was used to record and confirm the monoclonality of single cell clones. 24 single clones with higher titer and good confluency were selected. Finally, top clone showing the best productivity and growth performance was selected.

The selected top clone was subject to a fed-batch culture in a 3 L bioreactor with control and monitoring of variables such as pH, dissolved oxygen (DO), agitation and temperature. Top clone was cultured using the materials and reagents listed in Table 4 according to the GenScript's cell culture process development platform conditions and parameters. Culture supernatants were harvested on day 15 or when the viability was 70% or less, whichever occurs earlier.

TABLE 4
Materials and reagents for culture of top clone
for GLB1S protein expression in 3 L scale

| Culture Medium/Reagent | Manufacturer/Brand | Catalog No. | Abbreviation |
|---|---|---|---|
| Gibco ™Dynamis ™AGT ™ | ThermoFisher | A26175-02 | Dynamis |
| Medium | | | |
| Gibco ™EfficientFeed ™B+ | ThermoFisher | A25030-01 | Feed B+ |
| AGT ™ Supplement | | | |
| HyClone ™Cell Boost ™7a | Cytiva | SH31026.01 | 7a |
| HyClone ™Cell Boost ™7b | Cytiva | SH31027.02CN | 7b |
| D-(+)-Glucose | Merck | 1.37048.5000 | Gluc |
| Sodium Dextran Sulfate 5000 | FUJIFILM | 194-13402 | DSS |
| Poloxamer P188 | Merck | 1.37112.1000 | P188 |
| Na₂CO₃ | Merck | 1.06398.5000 | Na₂CO₃ |
| FoamAway Irradiated AOF | ThermoFisher | A1036902 | Foam Away |
| NaOH | Merck | 1.06482.5000 | NaOH |

Titers of cell culture supernatants harvested were measured to be 2.409 g/L using the 4-MU-β-galactopyranoside (MUG) assay.

The selected top clone was then subject to a fed-batch culture in a 50 L bioreactor with control and monitoring of variables such as pH, dissolved oxygen (DO), agitation and temperature, in order to demonstrate process scalability. Top clone was cultured using the materials and reagents listed in Table 5 according to the GenScript's cell culture process development platform conditions and parameters. Culture supernatants were harvested on day 15 or when the viability was 70% or less, whichever occurs earlier.

TABLE 5
Materials and reagents for culture of top clone
for GLB1S protein expression in 50 L scale

| Culture Medium/Reagent | Manufacturer/Brand | Catalog No. | Abbreviation |
|---|---|---|---|
| Gibco ™Dynamis ™AGT ™ | ThermoFisher | A26175-03 | Dynamis |
| Medium | | | |
| Gibco ™EfficientFeed ™B+ | ThermoFisher | A25030-01 | Feed B+ |
| AGT ™ Supplement | | | |
| HyClone ™Cell Boost ™7a | Cytiva | SH31026.05 | 7a |
| HyClone ™Cell Boost ™7b | Cytiva | SH31027.04CN | 7b |
| D-(+)-Glucose | SIGMA | SLCK0148 | Gluc |
| Sodium Dextran Sulfate 5000 | FUJIFILM | 194-13402 | DSS |
| Poloxamer P188 | Merck | 1.37112.1000 | P188 |
| FoamAway Irradiated AOF | ThermoFisher | A1036902 | Foam Away |
| NaOH | Merck | 1.06482.5000 | NaOH |

Titers of cell culture supernatants harvested were measured to be 2.061 g/L using the 4-MU-β-galactopyranoside (MUG) assay.

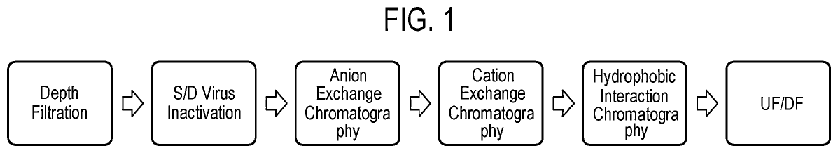

Further, the product harvested from the bioreactor was purified by developed purification process. After the supernatant of the culture was obtained, the proteins were purified in the order of depth filtration (DF), solvent/detergent(S/D), anion exchange chromatography (AEX), cation exchange chromatography (CEX), hydrophobic interaction chromatography (HIC) and ultrafiltration/diafiltration. The detailed purification work flow is shown in FIG. 1. The purified protein product was analyzed by RP-HPLC (reverse phase-high performance liquid chromatography) and SEC-HPLC (size exclusion-high performance liquid chromatography). The RP-HPLC and SEC-HPLC analysis results are shown in Table 6.

TABLE 6
RP-HPLC and SEC-HPLC Analysis Results of GLB1S Protein

| RP-HPLC (%) | | | SEC-HPLC (%) | | |
|---|---|---|---|---|---|
| Pre-Peak | Main peak | Post-Peak | HMW | Main peak | LMW |
| 0.43 | 99.57 | ND | 2.09 | 97.26 | 0.65 |

As shown in Table 6, the purity of the product as measured by RP-HPLC and SEC-HPLC was shown to be high. The main peak in RP-HPLC indicated the target protein GLB1S, and the pre-peak indicated fragments. The pre-peak ratio of the product as measured by RP-HPLC was shown to be very low. The main peak ratio (GLB1S) of the product as measured by SEC-HPLC was above 99%, and the high molecular weight (HMW) and low molecular weight (LMW) ratios of the product were acceptable as well.

### Example 3. Confirmation of GLB1S Protein Expression via SDS-PAGE and SEC-HPLC

GLB1S protein were obtained through 50 L culture and purification described in Example 2 above.

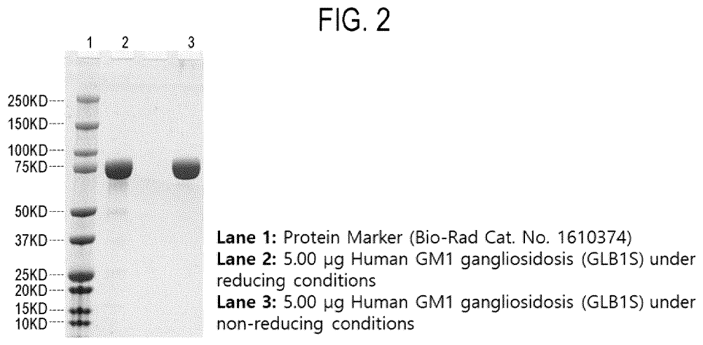

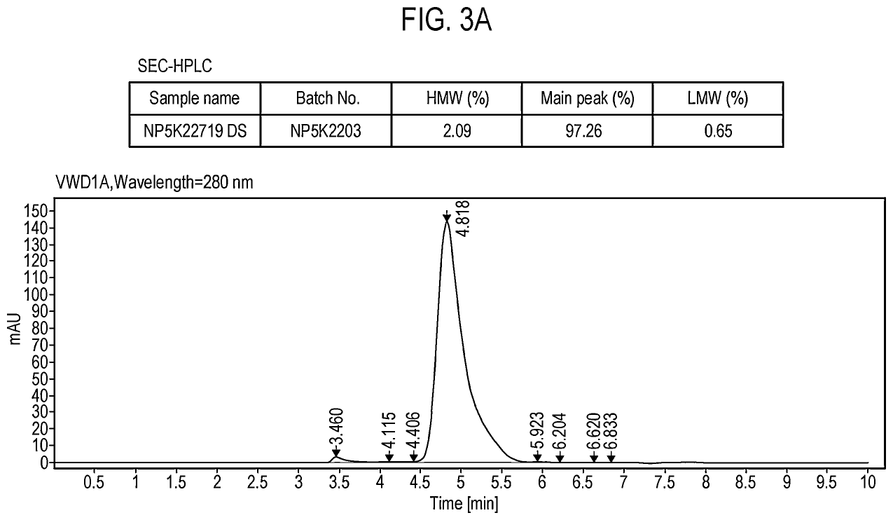

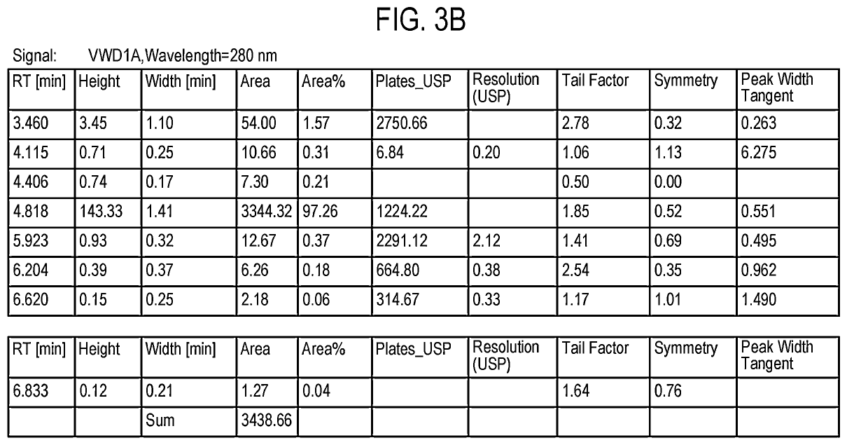

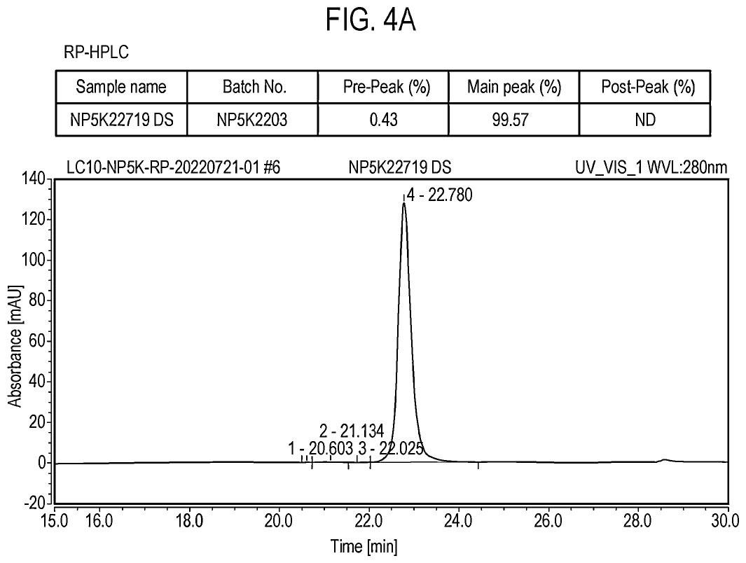

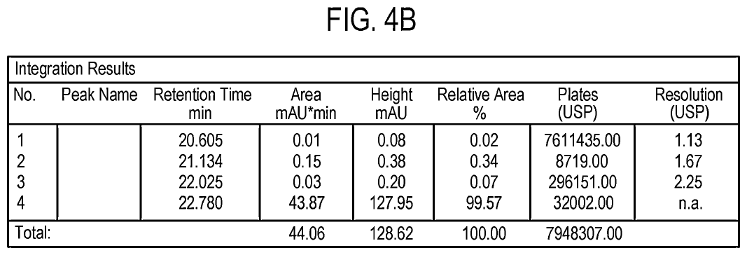

SDS-PAGE, SEC-HPLC and RP-HPLC analysis were conducted on the purified protein, and the results are shown in FIGS. 2, 3, and 4. As shown in FIG. 2, the GLB1S protein (with a molecular weight between marker of 50 kDa and marker of 75 kDa) was shown as the main product. As shown in FIG. 3A and 3B, the GLB1S protein of 97.26% purity was obtained. As shown in FIG. 4A and 4B, the RP-HPLC of GLB1S protein was 99.57%.

### Example 4. Confirmation of GLB1S Protein Activity

Enzymatic activity of GLB1S (rhGLB1S), rhGLB1 (GenScript) and rhGLB1-Fc (AbClon) was measured. In order to obtain rhGLB1S, CHO-K1 cells expressing GLB1S protein were cultured on a 10 L scale in the manner described in Examples 1 and 2 above, and the supernatant of the culture was purified in the order of anion exchange chromatography (AEX), cation exchange chromatography (CEX), hydrophobic interaction chromatography (HIC) and ultrafiltration/diafiltration. Specifically, 1 mg/ml of GLB1S solution was used for activity analysis. For rhGLB1-Fc, the hinge, CH2, and CH3 of the heavy chain of immunoglobulin gamma-1 (SEQ ID NO: 3) was used as Fc. The substrate solution of 0.5 mM 4-MU-β-galactopyranoside (MUG, 0.17 mg/ml) dissolved in 0.1M sodium acetate-acetic acid buffer (pH 4.0) containing 5.84 mg/ml sodium chloride (NaCl, 0.1M), which was stabilized at 4° C. or −20° C. for several months, was used.

This analysis required 0.020-0.030 mg of the protein for white blood cells and 0.010-0.020 mg of the protein for fibroblasts and amniotic fluid cells. In order to achieve the required protein concentration, 0.10 ml of diluted tissue lysate was placed in 12×75 mm tubes, and duplicates for each sample were prepared. Then, 0.10 ml of the above substrate solution was added to the tubes to initiate the reaction, and the tubes were incubated at 37° C. for 20 minutes. The reaction was then stopped by the addition of 1.30 mL of 0.17M glycine-carbonate buffer (pH 9.8). For blanks (blank tubes), only the substrate solution was cultured, and then the reaction was stopped by adding glycine-carbonate buffer. Then, a control lysate formulation diluted to an appropriate protein concentration was added. Fluorescence values were read at excitation wavelength of 360 nm and emission wavelength of 415 nm for the prepared samples, and the duplicate readings were averaged. The activity was calculated via the following equation, and the results are shown in Table 7.

$$Activity ( n mol / hr / mg ) = \frac{\begin{matrix}( Fluorescence of Sample - \\ Fluorescence of Blank Tube ) \times 3\end{matrix}}{\begin{matrix}mg of protein per analysis \times \\ Fluorescence of 1 n mol 4 - MU\end{matrix}} \qquad ( Equation 1 )$$

TABLE 7
Enzymatic activity of GLB1, GLB1S and GLB1-Fc

| Protein | 1:1000 Dilution | 1:2000 Dilution |
|---|---|---|
| rhGLB1S | 416,953 | 486,007 |
| rhGLB1 | 371,604 | 436,399 |
| rhGLB1-Fc | 11,946 | — |

As shown in Table 7, at 1:1000 dilution, the activity of rhGLB1S protein according to the present disclosure was measured to be 416,953 nmol/hr/mg, increased by about 12.2% compared to that of the full-length rhGLB1 protein (371,604 nmol/hr/mg). At 1:2000 dilution, the activity of rhGLB1S protein was measured to be about 11.4% higher than that of rhGLB1 protein. In other words, the activity of GLB1S protein, which is a truncated version of GLB1 protein (i.e., truncated at the C-terminus), was shown to be higher than that of the full-length GLB1 protein or the antibody-conjugated GLB1 protein (GLB1-Fc). The data above show that GLB1S protein can be used as a drug with equivalent efficacy (compared to GLB1 protein) for the treatment, prevention or amelioration of GM1 gangliosidosis or Morquio syndrome B.

### Example 5. Preparation of GLB1 KO (knock-out) Mouse Model

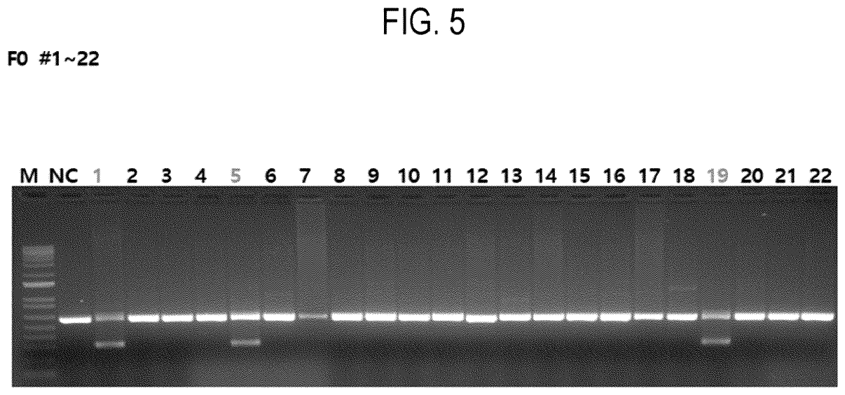

GLB1 KO mouse model was prepared using Clustered Regularly Interspaced Short Palindromic Repeats (CRISPR) gene editing technology. Exon 6 of the mouse GLB1 gene was targeted for editing, and such preparation was carried out by Macrogen. To confirm the knock-out (KO) status of the GM1 gangliosidosis or Morquio syndrome B model mice (GLB1 KO mice), genotyping was performed. 22 F0 transgenic mice were screened for GLB1 mutations, and it was determined that the KO mutation was present in mice F0 #1, #5, and #19. Among these, F0 #1 was selected and bred for subsequent experiments (FIG. 5).

### Example 6. Confirmation of the Effects of GLB1S Administration in GLB1 KO Mouse Model

### 1. Control Groups and Test Groups

TABLE 8
Control groups and test groups

| | | No. of | Administration | Administration | Administration |
|---|---|---|---|---|---|
| Group | Sex | mouse | dose level (mg/kg) | dose volume (ml/kg) | substance |
| G1 | M/F | 15 | — | 2 | Vehicle |
| G2 | M/F | 15 | — | 2 | Vehicle |
| G3 | M/F | 15 | 0.1 | 2 | GLB1S (0.05 mg/ml) |
| G4 | M/F | 15 | 0.5 | 2 | GLB1S (0.25 mg/ml) |
| G5 | M/F | 15 | 1 | 2 | GLB1S (0.5 mg/ml) |
| G6 | M/F | 15 | 2 | 2 | GLB1S (1 mg/ml) |
| G7 | M/F | 15 | 4 | 2 | GLB1S (2 mg/ml) |

G1: Wild type mouse group (wild type control group);
G2: KO mouse group (negative control group);
G3-G7: KO mouse group (test group)

### 2. Administration of Vehicle or GLB1S

The healthy mice were weighed, ranked, and then randomly assigned to groups according to Table 8. This was done to ensure that the average weight and sex distribution were as evenly distributed as possible across the groups.

Following the restraint of the mice with a restraint device or retaining appliance, vehicle or GLB1S was administered intravenously once using an insulin syringe (single administration).

### 3. Assessment of Body and Organs

### 3-1. Assessment Items and Sampling

General symptoms and body weight of the mice were assessed after the administration of vehicle or GLB1S. From the administration and throughout the observation periods, mortality and the type and severity of general symptoms were observed and recorded for each mouse at least once a day. The day of administration was designated as Day 0.

Body weight of mouse was measured at various time points, including at the time of mouse acquisition, group separation, administration of vehicle or GLB1S, necropsy, and once per week during the observation period.

As to sampling, five mice were sampled on each of Day 7, Day 14 and Day 28 after the administration of vehicle or GLB1S. The mice were perfused on the scheduled necropsy day, followed by necropsy to harvest organs. The harvested samples were subsequently stored at −70° C. Since one of the mice was dropped out from the group G3, the experiment for G3 was completed with 14 mice (i.e., 5 mice sampled on each of Day 7 and Day 14, and 4 mice sampled on Day 28).

### 3-2. Statistical Analysis

Statistical analysis was conducted using SPSS Statistics 12.0K for medical science. Comparisons were made between the normal control group (G1) and the negative control group (G2), as well as between the negative control group (G2) and the test groups (G3-G7). Since the number of mice in each group was 5 or fewer, non-parametric comparison of Kruskal-Wallis followed by Mann-Whitney post hoc tests with Bonferroni correction was conducted. Statistical significance was determined at a level of p<0.05 for all analyses.

### 3-3. Assessment Results

### (1) Incidence of Mortality

A mouse in the 0.1 mg/kg dose group (G3) was found dead on Day 27. However, no other mortalities were observed in the remaining test groups (G1, G2, G4, G5, G6 and G7).

### (2) General Symptoms

None of the groups (G1-G7) exhibited adverse effects.

### (3) Body Weight

There were no differences in body weight observed among all the groups (G1-G7) from the dosing and throughout the observation period.

TABLE 9
Body weight
BODY WEIGHT (g)

| GROUP | DAY 0 | DAY 7 | DAY 14 | DAY 21 | DAY 28 |
|---|---|---|---|---|---|
| G1 | 16.09 ± 0.33 | 18.63 ± 0.51 | 20.98 ± 0.65 | 21.23 ± 0.78 | 23.03 ± 0.94 |
| G2 | 14.91 ± 0.68 | 18.67 ± 0.69 | 20.90 ± 0.79 | 23.09 ± 1.15 | 24.05 ± 1.02 |
| G3 | 15.77 ± 0.54 | 18.41 ± 0.86 | 19.55 ± 1.09 | 20.31 ± 2.16 | 22.62 ± 2.32 |
| G4 | 14.98 ± 0.53 | 18.34 ± 0.56 | 20.62 ± 0.92 | 21.92 ± 0.91 | 23.02 ± 1.07 |
| G5 | 15.16 ± 0.84 | 18.07 ± 0.69 | 19.76 ± 0.95 | 21.42 ± 1.42 | 22.16 ± 1.42 |
| G6 | 15.22 ± 0.65 | 18.41 ± 0.68 | 20.77 ± 1.03 | 21.99 ± 1.82 | 22.82 ± 1.90 |
| G7 | 15.31 ± 0.51 | 18.53 ± 0.56 | 20.69 ± 0.76 | 21.34 ± 1.18 | 22.14 ± 1.41 |

Data were represented by mean ± S.E. The results were statistically analyzed by Kruskal-Wallis.

### (4) Organ Weight

No statistically significant differences in organ weights were observed among all test groups. However, on Day 7, the negative control group exhibited greater liver and spleen weights by more than 32% compared to those of the normal control group. All GLB1S-treated groups (G3-G7) showed less liver weight by more than 22% compared to that of the negative control group. All of the foregoing differences in liver weight and spleen weight were not statistically significant.

TABLE 10
Organ weights (Day 7)
ORGAN WEIGHT (g)

| GROUP | HEART | LIVER | SPLEEN |
|---|---|---|---|
| G1 | 0.098 ± 0.007 | 1.033 ± 0.137 | 0.059 ± 0.002 |
| G2 | 0.099 ± 0.005 | 1.363 ± 0.301 | 0.091 ± 0.008 |
| G3 | 0.105 ± 0.007 | 1.058 ± 0.111 | 0.067 ± 0.005 |
| G4 | 0.094 ± 0.004 | 1.007 ± 0.106 | 0.070 ± 0.010 |
| G5 | 0.101 ± 0.005 | 1.015 ± 0.148 | 0.079 ± 0.002 |
| G6 | 0.100 ± 0.004 | 0.918 ± 0.064 | 0.071 ± 0.006 |
| G7 | 0.099 ± 0.001 | 0.956 ± 0.054 | 0.080 ± 0.003 |
| GROUP | BRAIN | KIDNEY | LUNG |
| G1 | 0.443 ± 0.005 | 0.125 ± 0.008 | 0.209 ± 0.026 |
| G2 | 0.430 ± 0.012 | 0.129 ± 0.009 | 0.186 ± 0.015 |
| G3 | 0.446 ± 0.008 | 0.125 ± 0.008 | 0.175 ± 0.021 |
| G4 | 0.439 ± 0.007 | 0.128 ± 0.007 | 0.198 ± 0.029 |
| G5 | 0.431 ± 0.012 | 0.138 ± 0.010 | 0.175 ± 0.020 |
| G6 | 0.437 ± 0.009 | 0.137 ± 0.010 | 0.228 ± 0.027 |
| G7 | 0.433 ± 0.005 | 0.126 ± 0.002 | 0.205 ± 0.012 |

Data were represented by mean ± S.E. The results were statistically analyzed by Kruskal-Wallis.

### 4. Determination of Keratan Sulfate Content in Mouse Liver Tissue

### 4-1. Method for Determination of Keratan Sulfate Content

Keratan sulfate levels were assessed by collecting liver tissue from control and test groups on Day 7, Day 14 and Day 28 following the administration of vehicle or GLB1S. The explanations of the negative control and test groups and the number of mice in each group are shown in Table 11 below. The collected liver tissue from the mice underwent homogenization, removal of liver tissue lipids, and enzymatic treatment to produce keratan sulfate, which has disaccharide form. Subsequently, keratan sulfate was selectively extracted using PGC-SPE, and its content was quantified using LC-MS/MS.

TABLE 11
Information on liver tissue collection
for keratan sulfate measurement

| | Administration | | No. of | Sampling | |
|---|---|---|---|---|---|
| Group | substance | Dosage | mouse | point | |
| G2 | Vehicle | 2 ml/kg | 5 | 7 | D |
| | | | 5 | 14 | D |
| | | | 5 | 28 | D |
| G3 | GLB1S | 0.1 mg/kg:2 mL/kg | 5 | 7 | D |
| | | | 5 | 14 | D |
| | | | 4 | 28 | D |
| G4 | GLB1S | 0.5 mg/kg:2 mL/kg | 5 | 7 | D |
| | | | 5 | 14 | D |
| | | | 5 | 28 | D |
| G5 | GLB1S | 1 mg/kg:2 mL/kg | 4 | 7 | D |
| | | | 4 | 14 | D |
| | | | 4 | 28 | D |
| G6 | GLB1S | 2 mg/kg:2 mL/kg | 5 | 7 | D |
| | | | 5 | 14 | D |
| | | | 5 | 28 | D |
| G7 | GLB1S | 4 mg/kg:2 mL/kg | 3 | 7 | D |
| | | | 3 | 14 | D |
| | | | 3 | 28 | D |

### 4-2. Results on Keratan Sulfate Content in Mouse Liver Tissue

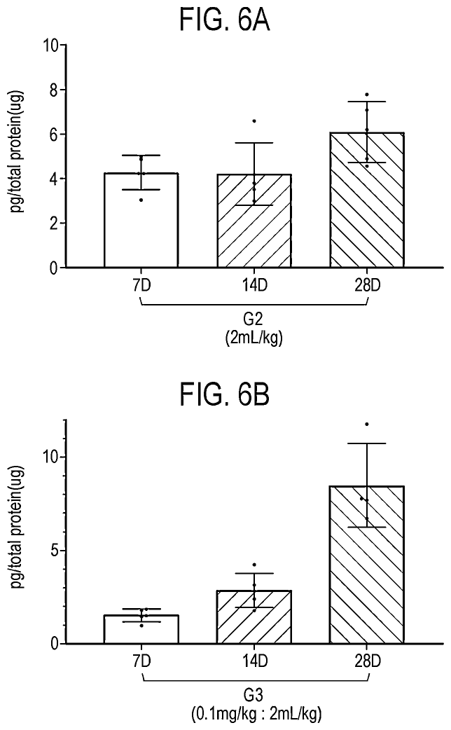

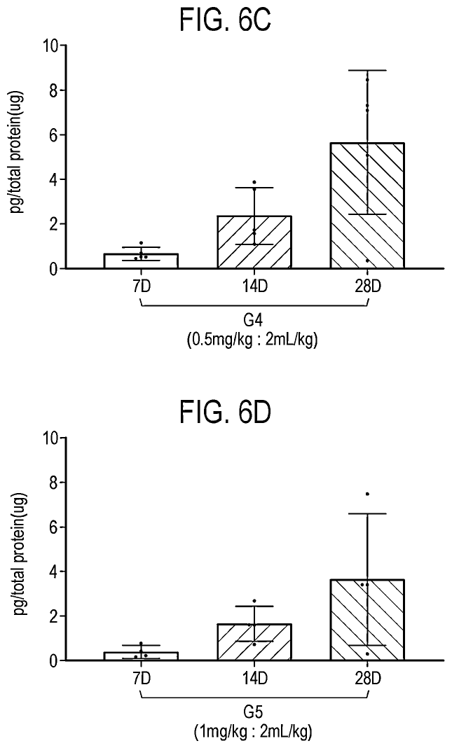

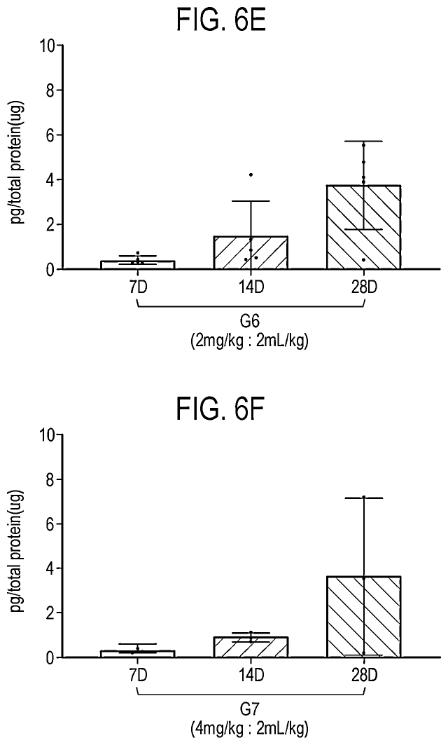

FIG. 6A-6F are graphs presenting the analysis results of keratan sulfate content of each group on Day 7, 14 and 28. As depicted in FIG. 6A-6F, the keratan sulfate content exhibited an increase over time in all groups, except for G2, which served as the negative control group. This indicates that the GLB1S can significantly inhibit keratan sulfate levels up to 14 days after GLB1S administration. At Day 28 following GLB1S administration, reaccumulation of keratan sulfate was observed in all treatment groups.

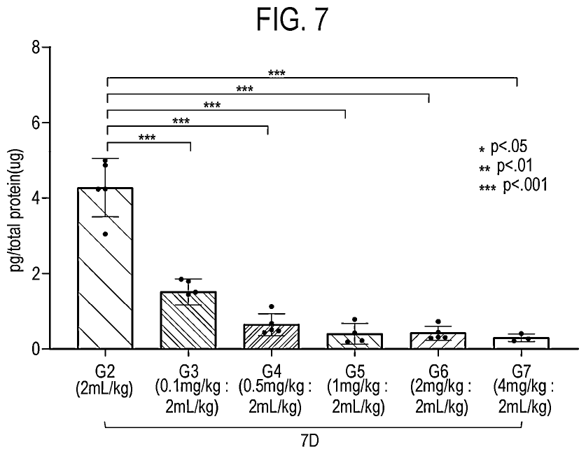

FIG. 7 is a graph demonstrating the results of the keratan sulfate content analysis after 7 days of vehicle or GLB1S administration. As depicted in FIG. 7, there was a significant dose-dependent decrease in keratan sulfate content in all GLB1S-treated groups compared to G2.

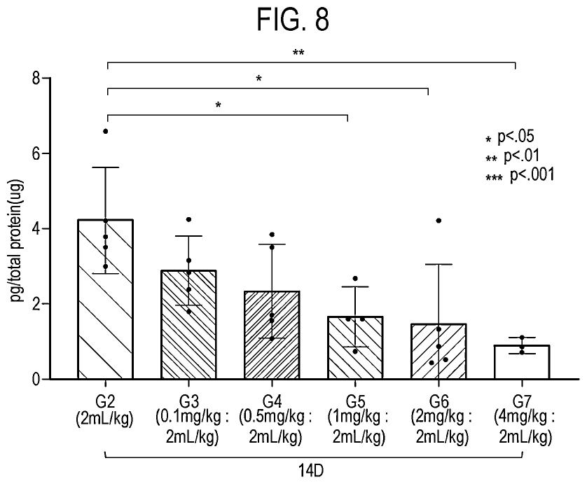

FIG. 8 is a graph presenting the results of the keratan sulfate content assay conducted 14 days after vehicle or GLB1S treatment. As shown in FIG. 8, a significant reduction in keratan sulfate content was observed in G5, G6, and G7, which correspond to the treatment groups receiving doses above 1.0 mg/kg, when compared to G2.

Based on the above results, a significant enzyme replacement therapy effect can be expected when GLB1S is administered on a weekly or biweekly basis.

In the case of rhGLB1 (a full-length protein), it is very difficult to obtain uniform proteins in the manufacturing process, due to heterogeneous protein truncation observed in rhGLB1. This becomes an obstacle to produce the protein for therapeutic use using large-scale manufacturing facilities. Additional treatment or purification processes after expressing the protein from transformed host cells may be needed in order to obtain the protein that meets the standard for drug marketing approval. The use of host cells expressing the full-length rhGLB1 protein, due to heterogeneity among the expressed proteins, does not ensure the predictable quality of the drug and this also raises drug safety concerns.

According to one embodiment of the present disclosure, GLB1S protein did not show problems of the full-length GLB1 proteins described above (e.g., heterogeneous protein truncation), and it has been confirmed for the first time that the GLB1S protein has equivalent enzymatic activity compared to the full-length GLB1 protein. The present disclosure demonstrated the production of GLB1S, which significantly improved the predictability of protein manufacturing conditions and drug quality on a commercial scale, and showed that the GLB1S protein can be used as a drug with better efficacy for the treatment, prevention or amelioration of GM1 gangliosidosis or Morquio syndrome B.

1. A protein comprising an amino acid sequence having at least 90% sequence identity of the amino acid sequence of SEQ ID NO: 1.

2. A protein of claim 1, comprising an amino acid sequence of SEQ ID NO: 1.

3. A polynucleotide encoding the protein of claim 1 or 2.

4. An expression vector comprising the polynucleotide of claim 3.

5. A host cell comprising the polynucleotide of claim 3 or the expression vector of claim 4.

6. A method of treating, preventing or ameliorating GM1 gangliosidosis or Morquio syndrome B comprising:
administering to a patient with GM1 gangliosidosis or Morquio syndrome B a therapeutically effective amount of a pharmaceutical composition comprising a protein of claim 1 or 2.

7. The method of claim 6, wherein after the administration of the therapeutically effective amount of the pharmaceutical composition, at least one symptom of GM1 gangliosidosis or Morquio syndrome B is reduced in intensity, severity or frequency, or has delayed onset.

8. The method of claim 6, wherein the therapeutically effective amount of the pharmaceutical composition is administered in a dose of 0.1 mg/kg to 100 mg/kg.

9. The method of claim 6, wherein the therapeutically effective amount of the pharmaceutical composition is administered via an intracerebroventricular (ICV) administration or an intravenous (IV) administration.

10. The method of claim 9, wherein the ICV administration comprises administering to the patient the therapeutically effective amount of the pharmaceutical composition via an intraventricular catheter system comprising a reservoir and a catheter connected to the reservoir.

11. The method of claim 6, wherein the protein has equivalent enzymatic activity to a full-length GLB1 protein.

12. The method of claim 6, wherein the therapeutically effective amount of the pharmaceutical composition is configured to increase GLB1 enzymatic activity in a blood of the patient by at least 50%.

13. The method of claim 6, wherein the therapeutically effective amount of the pharmaceutical composition is administered once a week, once every two weeks, twice a month or once a month.

14. The method of claim 6, wherein the therapeutically effective amount of the pharmaceutical composition reduces the amount of keratan sulfate in one or more internal organs of the patient.

15. A pharmaceutical composition for treating, preventing or ameliorating GM1 gangliosidosis or Morquio syndrome B, comprising a protein of claim 1 or 2.

16. The pharmaceutical composition of claim 15, wherein the protein has equivalent enzymatic activity to a full-length GLB1 protein.

17. The pharmaceutical composition of claim 15, wherein the pharmaceutical composition is configured to increase GLB1 enzymatic activity in a blood of the patient by at least 50%.

18. The pharmaceutical composition of claim 15, wherein the pharmaceutical composition reduces the amount of keratan sulfate in one or more internal organs of the patient.

19. A method of producing a protein comprising an amino acid sequence of SEQ ID NO: 1, comprising:
a) introducing a target gene encoding a protein comprising an amino acid sequence of SEQ ID NO: 1 into a vector;
b) transfecting a host cell with the vector;
c) culturing the transfected cell and harvesting cell culture supernatants; and
d) purifying the supernatant to obtain the protein.

20. The method of claim 19, wherein the host cell is a Chinese hamster ovary (CHO) cell.

21. A protein produced by the method of claim 19 or 20.
